(19) United States
(12) Patent Application Publication
(10) Pub. No.: US 2006/0024258 A1
(43) Pub. Date: Feb. 2, 2006

(54) Water-in-oil emulsion comprising a non-volatile non-silicone oil, cationic and nonionic surfactants, and an alkylmonoglycoside or alkylpolyglycoside

(75) Inventors: Geraldine Fack, Levallois-Perret (FR); Chrystel Pourille-Grethen, Clichy (FR)

Correspondence Address: OBLON, SPIVAK, MCCLELLAND, MAIER & NEUSTADT, P.C., 1940 DUKE STREET, ALEXANDRIA, VA, 22314, US

(73) Assignee: L'OREAL, Paris (FR)

(21) Appl. No.: 11/192,083

(22) Filed: Jul. 29, 2005

Related U.S. Application Data
(60) Provisional application No. 60/609,828, filed on Sep. 15, 2004.

(30) Foreign Application Priority Data
Aug. 2, 2004 (FR) 0408537

(51) Int. Cl. A61K 8/97; A61K 8/41

(52) U.S. Cl. 424/70.4; 424/70.28; 424/74

(57) ABSTRACT

A hair composition in the form of an water-in-oil emulsion, containing at least one non-volatile non-silicone oil, at least one cationic surfactant, at least one nonionic surfactant, and from 0.01% to 10% by weight relative to the total weight of the composition of at least one material selected from (C₁₂₋₃₀ alkyl)monoglycosides and (C₁₂₋₃₀ alkyl)polyglycosides.

### REFERENCE TO PRIOR APPLICATIONS

This application claims priority to U.S. provisional application 60/609,828 filed Sep. 15, 2004, and to French patent application 04 08537 filed Aug. 2, 2004, both incorporated herein by reference.

### FIELD OF THE INVENTION

The present invention relates to a cosmetic hair treatment composition, of water-in-oil emulsion type, comprising, in a cosmetically acceptable medium, at least one non-volatile non-silicone oil, at least one cationic surfactant, at least one nonionic surfactant and from 0.01% to 10% by weight relative to the total weight of the composition of at least one of an alkylmonoglycoside and a alkylpolyglycoside, and to a cosmetic hair treatment process.

### BACKGROUND OF THE INVENTION

Water-in-oil emulsions, especially those free of silicone compounds, are commonly used in cosmetics and in particular for skincare since they allow a lipid film to be formed on the surface of the skin, thus protecting it against external attack and preventing transepidermal water loss.

However, these emulsions are used very little in the haircare field, and in particular in the field of conditioning of the hair. The reason for this is that they have two major drawbacks, namely that of not being able to be rinsed out easily and completely, leading to an unaesthetic greasy residue, and that of not allowing good conditioning of the hair to be obtained. Specifically, dull, sticky and dirty hair is generally obtained, and softening of the hair fibre is observed. The hair is also difficult to disentangle.

### SUMMARY OF THE INVENTION

The inventors have discovered, surprisingly, that the introduction of a particular combination of surfactants into a water-in-oil emulsion makes it possible not only to improve its rinseability and to obtain good cosmetic properties, but also to obtain a stable water-in-oil emulsion. This particular combination comprises at least one cationic surfactant, at least one nonionic surfactant, and from 0.01% to 10% by weight relative to the total weight of the composition of at least one of a (C₁₂₋₃₀ alkyl)monoglycoside and a (C₁₂₋₃₀ alkyl)polyglycoside.

### DETAILED DESCRIPTION OF THE PREFERRED EMBODIMENTS

One object of the present invention is thus a composition, of water-in-oil emulsion type, comprising, in a cosmetically acceptable medium, at least one non-volatile non-silicone oil, at least one cationic surfactant, at least one nonionic surfactant, and from 0.01% to 10% by weight relative to the total weight of the composition of at least one (C₁₂₋₃₀ alkyl)monoglycoside, at least one (C₁₂₋₃₀ alkyl)polyglycoside, or a mixture thereof.

Another object of the invention is a cosmetic hair treatment process using a composition according to the invention as described herein.

An object of the invention is also the use of the composition according to the invention for conditioning the hair, and especially as a hair conditioner.

Other objects, characteristics, aspects and advantages of the invention will emerge even more clearly on reading the description and the examples that follow.

According to the invention, a preferred cosmetic hair treatment composition, of water-in-oil emulsion type, comprises, in a cosmetically acceptable medium, at least one non-volatile non-silicone oil, at least one cationic surfactant, at least one nonionic surfactant and from 0.01% to 10% by weight relative to the total weight of the composition of at least one alkylmonoglycoside or alkylpolyglycoside.

The phrase “from 0.01% to 10% by weight relative to the total weight of the composition of at least one alkylmonoglycoside or alkylpolyglycoside” includes more than one alkylmonoglycoside, more than one alkylpolyglycoside, and mixtures of alkylmonoglycoside(s) and alkylpolyglycoside(s).

The term “cosmetically acceptable medium” means a medium that is compatible with the hair.

The term “oil” means any non-aqueous medium that is liquid at room temperature (25° C.±3° C.) and atmospheric pressure, with a water solubility at 25° C. of less than 0.5%.

The term “non-volatile oil” means an oil with a vapour pressure at room temperature (25° C.±3° C.) of less than 2.66 Pa (0.02 mmHg).

The non-volatile non-silicone oils that may be used in the present invention include those chosen from plant oils, animal oils, mineral oils, synthetic oils and fatty acid esters, and mixtures thereof.

Plant oils that may especially be mentioned include sweet almond oil, avocado oil, castor oil, olive oil, liquid jojoba wax, sunflower oil, wheatgerm oil, sesame seed oil, groundnut oil, grapeseed oil, soybean oil, rapeseed oil, safflower oil, coconut oil, corn oil, hazelnut oil, palm oil, apricot kernel oil and calophyllum oil.

An example of animal oil is perhydrosqualene.

The composition according to the invention may also comprise one or more mineral oils such as a liquid paraffin and petrolatum oil.

The composition according to the invention may also comprise one or more synthetic oils such as squalane, poly(α-olefins), for instance isododecane or isohexadecane, transesterified plant oils and fluorinated oils.

The composition according to the invention may also comprise one or more fatty esters, for instance the compounds of formula RₐCOOR_{b} in which Rₐ represents a linear or branched, hydroxylated or non-hydroxylated, saturated or unsaturated fatty acid residue containing from 4 to 29 carbon atoms and R_{b} represents a linear or branched, saturated or unsaturated hydrocarbon-based chain containing from 3 to 30 carbon atoms, the total number of carbon atoms in the ester being greater than 10. Examples that may especially be mentioned include purcellin oil (stearyl octanoate), isopropyl myristate, isopropyl palmitate, butyl stearate, hexyl laurate, isononyl isononanoate, 2-ethylhexyl palmitate, 2-hexyldecyl laurate, 2-octyldecyl palmitate, 2-octyldodecyl myristate, isostearyl neopentanoate and tridecyl neopentanoate.

The oils that are particularly preferred in the composition according to the invention are especially chosen from avocado oil, isododecane, isopropyl myristate and liquid jojoba wax.

The oil(s) as described above is (are) especially present in the composition according to the invention in an amount ranging from 0.1% to 30% by weight, preferably from 1% to 20% by weight and better still from 5% to 15% by weight relative to the total weight of the composition.

The composition according to the invention comprises one or more cationic surfactants, such as for example polyoxyalkylenated primary, secondary or tertiary fatty amine salts, quaternary ammonium salts, and mixtures thereof.

Examples of quaternary ammonium salts that may especially be mentioned include:
those corresponding to the general formula (I) below: 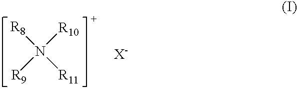
   in which the radicals R₈ to R₁₁, which may be identical or different, represent a linear or branched aliphatic radical containing from 1 to 30 carbon atoms, or an aromatic radical such as aryl or alkylaryl. The aliphatic radicals may comprise hetero atoms such as oxygen, nitrogen, sulfur and halogens. The aliphatic radicals are chosen, for example, from C₁₋₃₀ alkyl, C₁₋₃₀ alkoxy, C₂-C₆ polyoxyalkylene, C₁₋₃₀ alkylamide, (C₁₂-C₂₂)alkylamido(C₂-C₆)alkyl, (C₁₂-C₂₂)alkyl-acetate and C₁₋₃₀ hydroxyalkyl radicals; X⁻ is an anion chosen from the group of halides, phosphates, acetates, lactates, (C₂-C₆)alkyl sulfates and alkyl- or alkylaryl-sulfonates.

Among the quaternary ammonium salts of formula (I), those that are preferred are, firstly, tetraalkylammonium chlorides, for instance dialkyldimethylammonium or alkyltrimethylammonium chlorides in which the alkyl radical contains from about 12 to 22 carbon atoms, in particular behenyltrimethylammonium chloride, distearyldimethylammonium chloride, cetyltrimethylammonium chloride or benzyldimethylstearylammonium chloride, or, secondly, palmitylamidopropyltrimethylammonium chloride or stearamidopropyldimethyl(myristyl acetate)ammonium chloride sold under the name Ceraphyl® 70 by the company Van Dyk;
quaternary ammonium salts of imidazoline, for instance those of formula (II) below: 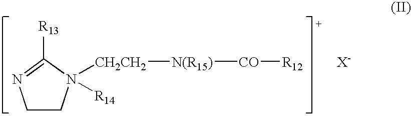
   in which R₁₂ represents an alkenyl or alkyl radical containing from 8 to 30 carbon atoms, for example tallow fatty acid derivatives, R₁₃ represents a hydrogen atom, a C₁-C₄ alkyl radical or an alkenyl or alkyl radical containing from 8 to 30 carbon atoms, R₁₄ represents a C₁-C₄ alkyl radical, R₁₅ represents a hydrogen atom or a C₁-C₄ alkyl radical, and X⁻ is an anion chosen from the group of halides, phosphates, acetates, lactates, alkyl sulfates, alkylsulfonates and alkylarylsulfonates. R₁₂ and R₁₃ preferably represent a mixture of alkenyl or alkyl radicals containing from 12 to 21 carbon atoms, for example tallow fatty acid derivatives, R₁₄ represents a methyl radical and R₁₅ represents a hydrogen atom. Such a product is sold, for example, under the name Rewoquat® W 75 by the company Rewo;
the quaternary diammonium salts of formula (III): 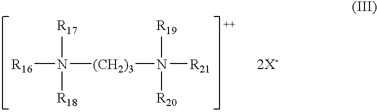
   in which R₁₆ represents an aliphatic radical containing from about 16 to 30 carbon atoms, R₁₇, R₁₈, R₁₉, R₂₀ and R₂₁, which may be identical or different, are chosen from a hydrogen atom and an alkyl radical containing from 1 to 4 carbon atoms, and X⁻ is an anion chosen from the group of halides, acetates, phosphates, nitrates and methyl sulfates. Such quaternary diammonium salts for example comprise propanetallowdiammonium dichloride;
quaternary ammonium salts containing at least one ester function, such as those of formula (IV) below: 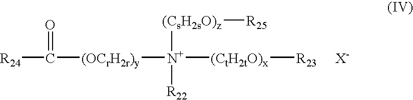
   in which:
R₂₂ is chosen from C₁-C₆ alkyl radicals and C₁-C₆ hydroxyalkyl or dihydroxyalkyl radicals;
R₂₃ is chosen from:
   a radical R₂₆ 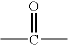
   linear or branched, saturated or unsaturated C₁-C₂₂ hydrocarbon-based radicals R₂₇,
   a hydrogen atom,
      R₂₅ is chosen from:
   a radical R₂₈ 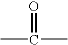
   linear or branched, saturated or unsaturated C₁-C₆ hydrocarbon-based radicals R₂₉,
   a hydrogen atom,
R₂₄, R₂₆ and R₂₈, which may be identical or different, are chosen from linear or branched, saturated or unsaturated C₇-C₂, hydrocarbon-based radicals;
r, s and t, which may be identical or different, are integers ranging from 2 to 6;
y is an integer ranging from 1 to 10;
x and z, which may be identical or different, are integers ranging from 0 to 10;
X⁻ is a simple or complex organic or inorganic anion;
with the proviso that the sum x+y+z is from 1 to 15, that when x is 0, then R₂₃ is R₂₇ and that when z is 0, then R₂₅ is R₂₉.

The alkyl radicals R₂₂ may be linear or branched, and more particularly linear.

Preferably, R₂₂ represents a methyl, ethyl, hydroxyethyl or dihydroxypropyl radical, and more particularly a methyl or ethyl radical.

Advantageously, the sum x+y+z is from 1 to 10.

When R₂₃ is a hydrocarbon-based radical R₂₇, it may be long and contain from 12 to 22 carbon atoms, or short and contain from 1 to 3 carbon atoms.

When R₂₅ is a hydrocarbon-based radical R₂₉, it preferably contains 1 to 3 carbon atoms.

Advantageously, R₂₄, R₂₆ and R₂₈, which may be identical or different, are chosen from linear or branched, saturated or unsaturated C₁₁-C₂₁ hydrocarbon-based radicals, and more particularly from linear or branched, saturated or unsaturated C₁₁-C₂₁ alkyl and alkenyl radicals.

Preferably, x and z, which may be identical or different, are 0 or 1.

Advantageously, y is equal to 1.

Preferably, r, s and t, which may be identical or different, are equal to 2 or 3 and even more preferably equal to 2.

The anion is preferably a halide (chloride, bromide or iodide) or an alkyl sulfate, more particularly methyl sulfate. However, methanesulfonate, phosphate, nitrate, tosylate, an anion derived from an organic acid, such as acetate or lactate, or any other anion that is compatible with the ammonium containing an ester function may be used.

The anion X⁻ is even more particularly chloride or methyl sulfate.

Use is made more particularly in the composition according to the invention of the ammonium salts of formula (IV) in which:
R₂₂ is a methyl or ethyl radical,
x and y are equal to 1;
z is equal to 0 or 1;
r, s and t are equal to 2;
R₂₃ is chosen from:
   a radical R₂₆ 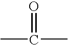
   methyl, ethyl or C₁₄-C₂₂ hydrocarbon-based radicals,
   a hydrogen atom;
R₂₅ is chosen from:
   a radical R₂₈ 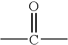
   a hydrogen atom;
R₂₄, R₂₆ and R₂₈, which may be identical or different, are chosen from linear or branched, saturated or unsaturated C₁₃-C₁₇ hydrocarbon-based radicals, and preferably from linear or branched, saturated or unsaturated C₁₃-C₁₇ alkyl and alkenyl radicals.

The hydrocarbon-based radicals are preferably linear.

Examples of compounds of formula (IV) that may be mentioned include the salts (especially chloride or methyl sulfate) of diacyloxyethyl-dimethylammonium, of diacyloxyethyl-hydroxyethyl-methylammonium, of monoacyloxyethyl-dihydroxyethyl-methyl-ammonium, of triacyloxyethyl-methylammonium, of monoacyloxyethyl-hydroxyethyl-dimethylammonium, and mixtures thereof. The acyl radicals preferably contain 14 to 18 carbon atoms and are more particularly derived from a plant oil, for instance palm oil or sunflower oil. When the compound contains several acyl radicals, these radicals may be identical or different.

These products are obtained, for example, by direct esterification of optionally oxyalkylenated triethanolamine, triisopropanolamine, alkyldiethanolamine or alkyldiisopropanolamine onto fatty acids or onto mixtures of fatty acids of plant or animal origin, or by transesterification of the methyl esters thereof. This esterification is followed by a quaternization using an alkylating agent such as an alkyl halide (preferably a methyl or ethyl halide), a dialkyl sulfate (preferably dimethyl or diethyl sulfate), methyl methanesulfonate, methyl para-toluenesulfonate, glycol chlorohydrin or glycerol chlorohydrin.

Such compounds are sold, for example, under the names Dehyquart® by the company Henkel, Stepanquat® by the company Stepan, Noxamium® by the company Ceca, and Rewoquat® WE 18 by the company Rewo-Witco.

The composition according to the invention preferably contains a mixture of quaternary ammonium mono-, di- and triester salts with a weight majority of diester salts.

Examples of mixtures of ammonium salts that may be used include the mixture containing 15% to 30% by weight of acyloxyethyl-dihydroxyethyl-methylammonium methyl sulfate, 45% to 60% of diacyloxyethyl-hydroxyethyl-methylammonium methyl sulfate and 15% to 30% of triacyloxyethyl-methylammonium methyl sulfate, the acyl radicals containing from 14 to 18 carbon atoms and being derived from optionally partially hydrogenated palm oil.

It is also possible to use the ammonium salts containing at least one ester function described in patents U.S. Pat. No. 4,874,554 and U.S. Pat. No. 4,137,180.

The cationic surfactants that are particularly preferred in the composition of the invention are chosen from quaternary ammonium salts, in particular from behenyltrimethylammonium chloride and cetyltrimethylammonium chloride.

The cosmetic hair treatment composition preferably comprises the cationic surfactant(s) in an amount ranging from 0.1% to 20% by weight, better still from 0.2% to 10% by weight and even more preferably from 0.5% to 8% by weight relative to the total weight of the composition.

The nonionic surfactants that may be used in the compositions of the present invention include those listed in “Handbook of Surfactants” by M. R. Porter, published by Blackie & Son (Glasgow and London), 1991, pp. 116-178). They are especially chosen from polyethoxylated, polypropoxylated or polyglycerolated alcohols, polyethoxylated, polypropoxylated or polyglycerolated α-diols, polyethoxylated, polypropoxylated or polyglycerolated (C₁₋₂₀)alkylphenols and polyethoxylated, polypropoxylated or polyglycerolated fatty acids, the fatty chain containing, for example, from 8 to 18 carbon atoms, the number of ethylene oxide or propylene oxide groups possibly ranging especially from 2 to 50 and the number of glycerol groups possibly ranging especially from 2 to 30.

Use may also be made of condensates of ethylene oxide and of propylene oxide with fatty alcohols; polyethoxylated fatty amides preferably containing from 2 to 30 ethylene oxide units, polyglycerolated fatty amides comprising on average from 1 to 5 and in particular from 1.5 to 4 glycerol groups, ethoxylated fatty acid esters of sorbitan containing from 2 to 30 ethylene oxide units, fatty acid esters of sucrose, fatty acid esters of polyethylene glycol, polyethoxylated plant oils, N—(C₆₋₂₄ alkyl)glucamine derivatives, and amine oxides such as (C₁₀₋₁₄ alkyl)amine oxides or N—(C₁₀₋₁₄ acyl)-aminopropylmorpholine oxides.

The nonionic surfactants are preferably not C₈-C₅₀ fatty alcohols.

The nonionic surfactants preferably used in the compositions of the invention have an HLB ranging from 1.5 to 10 and preferably from 1.5 to 7.

The HLB, or hydrophilic-lipophilic balance, of the nonionic surfactant(s) used according to the invention is the Griffin HLB defined in the publication J. Soc. Cosm. Chem. 1954 (Volume 5), pp. 249-256.

As examples of nonionic surfactants with an HLB ranging from 1.5 to 10, mention may be made especially of those sold under the following brand names:

Etocas 29 (HLB=1.7) by the company Croda, Genapol PF 10 (HLB=2) by the company Hoechst, Synperonic PE L81 (HLB=2) by the company ICI, Prox-Onic EP 1090-1 (HLB=3) by the company Protex, Sinnopal DPN2 (HLB=3.3) by the company Henkel, Antarox CA 210 (HLB=3.5) by the company Rhône-Poulenc, Antarox O1P (HLB=3.5) by the company Rhône-Poulenc, Alkasurf OP11 (HLB=3.6) by the company Rhône-Poulenc, Triton X15 (HLB=3.6) by the company Rohm & Haas, Alkasurf OP1 (HLB=3.6) by the company Rhône-Poulenc, Arlacel 121 (HLB=3.8) by the company ICI, Prox-Onic HR or HRH-05 (HLB=3.8) by the company Protex, Etocas 5 (HLB=3.9) by the company Hoechst, Genapol PF20 (HLB=4) by the company Hoechst, Imbentin N/7 A (HLB=4) by the company Kolb, Synperonic PE L122 (HLB=4) by the company ICI, Ethylan NP1 (HLB=4.5) by the company Harcros, Imbentin N/020 (HLB=4.5) by the company Kolb, Kotilen 0/3/020 (HLB=4.5) by the company Kolb, Synperonic PE L31 (HLB=4.5) by the company ICI, TO-55-A (HLB=4.5) by the company Hefti, Alkasurf NP-1 (HLB=4.6) by the company Rhône-Poulenc, Antarox CO 210 (HLB=4.6) by the company Rhône-Poulenc, Prox-Onic NP-1 (HLB=4.6) by the company Protex, Rhodiasurf NP2 (HLB=4.6) by the company Rhône-Poulenc, Soprophor BC2 (HLB=4.6) by the company Rhone-Poulenc, Triton N17 (HLB=4.6) by the company Rohm & Haas, Akyporox NP15 (HLB=4.7) by the company CHEM-Y, Texofor M2 (HLB=4.8) by the company Rhône-Poulenc, Alkasurf SA2 (HLB=4.9) by the company Rhône-Poulenc, Arlacel 989 (HLB=4.9) by the company ICI, Brij 72 (HLB=4.9) by the company ICI, Brij 92 (HLB=4.9) by the company ICI, Brij 93 (HLB=4.9) by the company ICI, Prox-Onic SA-1 or 2/02 (HLB=4.9) by the company Protex, Simulsol 72 (HLB=4.9) by the company SEPPIC, Simulsol 92 (HLB=4.9) by the company SEPPIC, Volpo S-2 (HLB=4.9) by the company Croda, Arlacel 581 (HLB=5.0) by the company ICI, Arlacel 582 (HLB=5.0) by the company ICI, Genapol 0-020 (HLB=5.0) by the company Hoechst, Imbentin POA/020 (HLB=5.0) by the company Kolb, and Mergital Q2 (HLB=5.0) by the company Henkel, Imbentin POA/024 (HLB=5.5) by the company ICI, Synperonic PE L92 (HLB=5.5) by the company ICI, Mergital LM2 (HLB=5.8) by the company Henkel, Atlas G-70140 (HLB=6) by the company ICI, Imbentin. AG/124S/020 (HLB=6) by the company Kolb, Imbentin. L/125/025 HLB=6 by the company Kolb, Simulsol 989 (HLB=6) by the company SEPPIC, Soprophor HR10 (HLB=6) by the company Rhône-Poulenc, Kotilen 0/1/050 (HLB=6.2) by the company Kolb, Croduret 10 (HLB=6.3) by the company Croda, Etocas 10 (HLB=6.3) by the company Croda, Imbentin OA/030 (HLB=6.3) by the company Kolb, Soprophor 208 (HLB=6.9) by the company Rhône-Poulenc, Ethylan 172 (HLB=7) by the company Harcros, Akyporox NP 40 (HLB=7.1) by the company Chem-Y, Polychol 5 (HLB=7.3) by the company Croda, Arlatone 985 (HLB=7.5) by the company ICI, Sandoxylate FOL4 (HLB=7.5) by the company Sandoz, Radiasurf 7453 (HLB=7.8) by the company Oleofina, Prox-onic OA-1/04 (HLB=7.9) by the company Protex, Prox-onic TD-1/03 (HLB=7.9) by the company Protex, Genapol PF 40 (HLB=8) by the company Hoechst, PGE-400-DS (HLB=8) by the company Hefti, PGE-400-DO (HLB=8) by the company Hefti, Sapogenat 6-040 (HLB=8) by the company Hoechst, Intrasol FA28/50/4 (HLB=8.1) by the company Stockhausen, Serdox NOG 200 S (HLB=8.5) by the company Servo, Berol 26 (HLB=8.9) by the company Berol Nobel, Genapol 0-050 (HLB=9) by the company Hoechst, Prox-onic LA-1/04 (HLB=9.2) by the company Protex, Eumulgin O5 (HLB=9.5) by the company Henkel, Etocas 20 (HLB=9.6) by the company Croda, Antarox CO 520 (HLB=10) by the company Rhône-Poulenc, Imbentin POA/060 (HLB=10) by the company Kolb, TO-55-EL (HLB=10) by the company Hefti.

The nonionic surfactants that are more particularly preferred are chosen from sorbitan isostearate, polyglyceryl isostearate, methyl-glucose dioleate, hydrogenated castor oil polyethoxylated with 7 mol of ethylene oxide, and mixtures thereof.

The nonionic surfactant(s) as defined above is (are) preferably contained in an amount ranging from 0.01% to 10% by weight, preferably from 0.1% to 5% by weight and better still from 0.2% to 3% by weight relative to the total weight of the composition.

The alkylmonoglycosides or alkylpolyglycosides that are particularly preferred in the invention are those in which the alkyl group contains from 16 to 24 carbon atoms.

A particularly preferred example that may especially be mentioned is arachidylglycoside.

The (C₁₂₋₃₀ alkyl)monoglycoside(s) or (C₁₂₋₃₀ alkyl)polyglycoside(s) is (are) contained in an amount ranging from 0.01% to 10% by weight, preferably from 0.02% to 5% by weight and more preferably from 0.05% to 1% by weight relative to the total weight of the composition.

Preferably, the weight ratio of oil(s)/nonionic surfactant(s) in the compositions of the invention is between 3 and 100, preferably between 10 and 75 and even more preferably between 15 and 40.

The cosmetically acceptable aqueous medium comprises water or a mixture of water and of a cosmetically acceptable solvent chosen from C₁-C₄ lower alcohols, such as ethanol, isopropanol, tert-butanol or n-butanol; polyols, for instance propylene glycol; polyol ethers; C₅-C₁₀ alkanes; C₃₋₄ ketones, for instance acetone and methyl ethyl ketone; C₁-C₄ alkyl acetates, for instance methyl acetate, ethyl acetate and butyl acetate; dimethoxyethane and diethoxyethane; and mixtures thereof.

The compositions according to the invention may also comprise at least one C₁₄₋₃₀ fatty alcohol, and preferably at least one alcohol chosen from myristyl alcohol, cetyl alcohol, stearyl alcohol, arachidyl alcohol, behenyl alcohol and erucyl alcohol. They are generally present in an amount of less than 10% by weight, preferably ranging from 0.01% to 5% by weight and more preferably from 0.05% to 1.5% by weight relative to the total weight of the composition.

The compositions according to the invention may also comprise at least one silicone oil that is well known in the art, in an amount of less than 10% by weight, preferably ranging from 0.01% to 8% and even more preferably from 0.1% to 5% by weight relative to the total weight of the composition.

Examples of silicone oils that may be used include linear or cyclic polydimethylsiloxanes.

The compositions according to the invention may also contain at least one additive such as a cationic, anionic, nonionic or amphoteric polymer; a natural or synthetic anionic, amphoteric, zwitterionic, nonionic or cationic, associative or non-associative polymeric thickener; a non-polymeric thickener, for instance an electrolyte or a sugar; a nacreous agent; an opacifier; a sunscreen; a fragrance; a dye; an organic or mineral particle; a preserving agent; a pH stabilizer.

A person skilled in the art will take care to select the optional additives and the amount thereof such that they preferably do not harm the properties of the compositions of the present invention. These additives are present in the composition according to the invention in an amount ranging for example from, e.g., 0 to 50% by weight relative to the total weight of the composition. Preferably, the compositions according to the invention contain less than 5% by weight of anionic surfactant, more preferably less than 1% by weight. Even more preferably, the compositions according to the invention do not contain any anionic surfactant.

The average emulsion particle size is preferably between 300 nanometers and 50 micrometers, more preferably between 500 nanometers and 20 micrometers, and even more preferably between 750 nanometers and 10 micrometers.

The compositions according to the invention may be in the form of fluid or thickened liquids, gels, creams, or simple or multiple emulsions.

The compositions may be used, for example, in shampoos, dyeing, bleaching or permanent-waving products, styling products, rinse-out care products, deep-care masks, shower gels, lotions or creams for treating the scalp, alternatively deposited onto wipes.

The present invention also relates to a cosmetic hair treatment process that comprises applying an effective amount of a composition as described above to the hair, and optionally rinsing it out after an optional leave-in time.

According to one preferred embodiment of the invention, the composition may be used for conditioning the hair and more particularly as a hair conditioner.

The examples that follow are given as illustrations of the present invention.

### EXAMPLES

The water-in-oil emulsions were prepared by mixing together the ingredients indicated in the table below in the indicated weight percentages relative to the total weight of the emulsion.

| | Ex. 1 | Ex. 2 | Ex. 3 |
|---|---|---|---|
| Isopropyl myristate | 9.25 | 9.3 | 8 |
| C₁₁₋₁₂ isoparaffin | — | — | 1.5 |
| Avocado oil | — | — | — |
| 3-Polyglyceryl isostearate and | 0.75 | — | — |
| sorbitan isostearate⁽¹⁾ | | | |
| Methylglucose dioleate⁽²⁾ | — | 0.7 | — |
| Hydrogenated castor oil of | — | — | 0.85 |
| polyethylene glycol⁽³⁾ | | | |
| Arachidylglucoside (at 15% AM)⁽⁴⁾ | 0.15 | 0.15 | 0.1 |
| | AM | AM | AM |
| Behenyltrimethylammonium chloride | 4 | 3 | 4 |
| (at 80% AM)⁽⁵⁾ | AM | AM | AM |
| Propylene glycol | — | 2 | 2 |
| Water qs | 100 | 100 | 100 |

AM: Active Material
⁽¹⁾sold under the trade name Arlacel 1690 by Uniqema
⁽²⁾sold under the trade name Glucate DO by Amerchol
⁽³⁾sold under the trade name Arlacel 989 by Uniqema
⁽⁴⁾sold under the trade name Montanov 202 by SEPPIC
⁽⁵⁾sold under the trade name Genamin KDMP by Clariant

AM: Active Material
⁽¹⁾: sold under the trade name Arlacel 1690 by Uniqema
⁽²⁾: sold under the trade name Glucate DO by Amerchol
⁽³⁾: sold under the trade name Arlacel 989 by Uniqema
⁽⁴⁾: sold under the trade name Montanov 202 by SEPPIC
⁽⁵⁾: sold under the trade name Genamin KDMP by Clariant

The various emulsions were applied to sensitized hair.

Good rinseability and good conditioning of the fibres were then observed.

The above written description of the invention provides a manner and process of making and using it such that any person skilled in this art is enabled to make and use the same, this enablement being provided in particular for the subject matter of the appended claims, which make up a part of the original description and including a composition in the form of an water-in-oil emulsion, comprising at least one non-volatile non-silicone oil, at least one cationic surfactant, at least one nonionic surfactant, and from 0.01% to 10% by weight relative to the total weight of the composition of at least one material selected from (C12-30 alkyl)monoglycosides and (C12-30 alkyl)polyglycosides. As used above, the phrases “selected from the group consisting of,” “chosen from,” “selected from,” and the like include mixtures of the specified materials.

All references, patents, applications, tests, standards, documents, publications, brochures, texts, articles, etc. mentioned herein are incorporated herein by reference. Where a numerical limit or range is stated, the endpoints are included. Also, all values and subranges within a numerical limit or range are specifically included as if explicitly written out. Terms such as “contain(s)” and the like as used herein are open terms meaning ‘including at least’ unless otherwise specifically noted.

The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein.

1. A composition in the form of an water-in-oil emulsion, comprising at least one non-volatile non-silicone oil, at least one cationic surfactant, at least one nonionic surfactant, and from 0.01% to 10% by weight relative to the total weight of the composition of at least one material selected from (C₁₂₋₃₀ alkyl)monoglycosides and (C₁₂₋₃₀ alkyl)polyglycosides.

2. The composition according to claim 1, wherein the non-volatile non-silicone oil is a plant oil, an animal oil, a mineral oil, a synthetic oil, a fatty acid ester, or a mixture thereof.

3. The composition according to claim 2, comprising a plant oil chosen from sweet almond oil, avocado oil, castor oil, olive oil, liquid jojoba wax, sunflower oil, wheatgerm oil, sesame seed oil, groundnut oil, grapeseed oil, soybean oil, rapeseed oil, safflower oil, coconut oil, corn oil, hazelnut oil, palm oil, apricot kernel oil and calophyllum oil.

4. The composition according to claim 2, comprising perhydrosqualene.

5. The composition according to claim 2, comprising a mineral oil chosen from liquid paraffin and liquid petrolatum oil.

6. The composition according to to claim 2, comprising a synthetic oil chosen from squalane, poly(α-olefins), transesterified plant oils and fluorinated oils.

7. The composition according to to claim 2, comprising a fatty ester chosen from purcellin oil, isopropyl myristate, isopropyl palmitate, butyl stearate, hexyl laurate, isononyl isononanoate, 2-ethylhexyl palmitate, 2-hexyldecyl laurate, 2-octyldecyl palmitate, 2-octyldodecyl myristate, isostearyl neopentanoate and tridecyl neopentanoate.

8. The composition according to claim 1, wherein the non-volatile non-silicone oil is chosen from avocado oil, isododecane, isopropyl myristate and liquid jojoba wax.

9. The composition according to claim 1, wherein it comprises oil(s) in an amount ranging from 0.1% to 30% by weight relative to the total weight of the composition.

10. The composition according to claim 1, wherein the cationic surfactants are chosen from optionally polyoxyalkylenated primary, secondary or tertiary fatty amine salts, quaternary ammonium salts, and mixtures thereof.

11. The composition according to claim 1, comprising at least one quaternary ammonium salts chosen from:
those corresponding to the general formula (I) below: 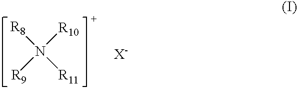
in which the radicals R₈ to R₁₁, which may be identical or different, represent a linear or branched aliphatic radical containing from 1 to 30 carbon atoms, or an aromatic radical; X⁻ is an anion chosen from the group of halides, phosphates, acetates, lactates, (C₂-C₆)alkyl sulfates and alkyl- or alkylaryl-sulfonates;
quaternary ammonium salts of imidazoline;
the quaternary diammonium salts of formula (III): 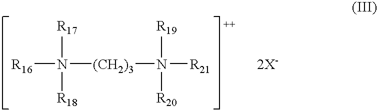
in which R₁₆ represents an aliphatic radical containing from about 16 to 30 carbon atoms, R₁₇, R₁₈, R₁₉, R₂₀ and R₂₁, which may be identical or different, are chosen from a hydrogen atom and an alkyl radical containing from 1 to 4 carbon atoms, and X⁻ is an anion chosen from the group of halides, acetates, phosphates, nitrates and methyl sulfates;
quaternary ammonium salts containing at least one ester function.

12. The composition according to claim 1, comprising at least one of behenyltrimethylammonium chloride and cetyltrimethylammonium chloride.

13. The composition according to claim 1, wherein it comprises cationic surfactant(s) in an amount ranging from 0.1% to 20% by weight relative to the total weight of the composition.

14. The composition according to claim 1, wherein the nonionic surfactant has an HLB ranging from 1.5 to 10.

15. The composition according to claim 14, wherein the HLB ranges from 1.5 to 7.

16. The composition according to claim 1, wherein the nonionic surfactant is chosen from polyethoxylated, polypropoxylated or polyglycerolated alcohols; polyethoxylated, polypropoxylated or polyglycerolated α-diols; polyethoxylated, polypropoxylated or polyglycerolated (C₁₋₂₀)alkylphenols; polyethoxylated, polypropoxylated or polyglycerolated fatty acids; condensates of ethylene oxide and of propylene oxide with fatty alcohols; polyethoxylated fatty amides; polyglycerolated fatty amides comprising on average from 1 to 5 glycerol groups; ethoxylated fatty acid esters of sorbitan containing from 2 to 30 ethylene oxide units; fatty acid esters of sucrose; fatty acid esters of polyethylene glycol; polyethoxylated plant oils; N-(C₆₋₂₄ alkyl)glucamine derivatives; amine oxides, and mixtures thereof.

17. The composition according to claim 16, wherein the nonionic surfactant is chosen from sorbitan isostearate, polyglyceryl isostearate, methylglucose dioleate, hydrogenated castor oil polyethoxylated with 7 mol of ethylene oxide, and mixtures thereof.

18. The composition according to claim 1, wherein it comprises the nonionic surfactant(s) in an amount ranging from 0.01% to 10% by weight relative to the total weight of the composition.

19. The composition according to claim 1, comprising at least one (C₁₂₋₃₀ alkyl)monoglycoside or (C₁₂₋₃₀ alkyl)polyglycoside in which the alkyl group contains from 16 to 24 carbon atoms.

20. The composition according to claim 1, wherein it comprises the (C₁₂₋₃₀ alkyl)monoglycoside(s) and (C₁₂₋₃₀ alkyl)polyglycoside(s) in an amount ranging from 0.02% to 5% by weight relative to the total weight of the composition.

21. The composition according to claim 1, wherein the weight ratio of oil(s)/nonionic surfactant(s) is between 3 and 100.

22. The composition according to claim 1, further comprising at least one C₁₄₋₃₀ fatty alcohol.

23. The composition according to claim 22, wherein the C₁₄₋₃₀ fatty alcohol is chosen from myristyl alcohol, cetyl alcohol, stearyl alcohol, arachidyl alcohol, behenyl alcohol, erucyl alcohol and mixtures thereof.

24. The composition according to claim 22, wherein it comprises the fatty alcohol(s) in an amount of less than 10% by weight relative to the total weight of the composition.

25. The composition according to claim 1, further comprising at least one cosmetically acceptable solvent.

26. The composition according to claim 25, wherein the cosmetically acceptable solvent is chosen from C₁-C₄ lower alcohols, polyols, polyol ethers, C₅-C₁₀ alkanes, C₃₋₄ ketones, C₁-C₄ alkyl acetates, dimethoxyethane, diethoxyethane, and mixtures thereof.

27. The composition according to claim 1, further comprising at least one silicone oil in an amount of less than 10% by weight relative to the total weight of the composition.

28. The composition according to claim 1, further comprising at least one additive chosen from cationic, anionic, nonionic or amphoteric polymers; natural or synthetic, anionic, amphoteric, zwitterionic, nonionic or cationic, associative or non-associative polymeric thickeners; non-polymeric thickeners; nacreous agents; opacifiers; sunscreens; fragrances; dyes; organic or mineral particles; preserving agents; pH stabilizers, and mixtures thereof.

29. A hair treatment process, comprising applying to the hair the composition of claim 1.

30. The method of claim 29, wherein said composition is a conditioner.
